(19) United States
(12) Patent Application Publication
(10) Pub. No.: US 2025/0120854 A1
(43) Pub. Date: Apr. 17, 2025

(54) Compression Bandage Systems with Areas of Increased Local Pressure

(71) Applicant: Solventum Intellectual Properties Company, Maplewood, MN (US)

(72) Inventors: Guido Hitschmann, Neuss (DE); Lynn M. Peterson, Chippewa Falls, WI (US)

(21) Appl. No.: 18/688,804

(22) PCT Filed: Sep. 8, 2022
(86) PCT No.: PCT/IB2022/058473

§ 371 Date: Mar. 4, 2024

Related U.S. Application Data
(60) Provisional application No. 63/242,715, filed on Sep. 10, 2021.

(51) Int. Cl. A61F 13/02

(52) U.S. Cl. CPC A61F 13/0273

(57) ABSTRACT

Bandages, compression bandage systems, and kits including the same are described. Further described methods for reducing or preventing swelling in an extremity with the use of bandages and compression bandage systems.

### BACKGROUND

Compression therapy has many pathophysiological benefits. It has been shown to reduce formation of excess interstitial fluid by opposing fluid filtration from blood capillaries into the tissue thus decreasing lymphatic load, shift fluid into uncompressed areas with functional lymphatics, increase lymphatic reabsorption and stimulation of lymphatic contractions, and increase lymphangion function. Compression therapy has been further shown to enhance muscle pump resulting in increased frequency and amplitude of lymph collector contractions, reduce venous reflux and venous hypertension, improve venous return, reduce elevated matrix metalloproteinase levels to promote healing of venous leg ulcers. Increasing arterial and pulsatile blood flow, reducing skin blood perfusion, reducing inflammatory response by release of anti-inflammatory mediators, downregulating proinflammatory cytokines, increasing fibrinolytic activity, and removing excess fluid to suppress proliferation of keratinocytes, fibroblasts, and endothelial cells are all further benefits of compression. For such reasons, compression therapy is a cornerstone for managing acute and chronic edema, which may result from a variety of ailments such as venous ulcers, congestive heart failure, and lymphedema.

Therapeutic compression stockings and wraps are worn over an affected extremity and compression (i.e., pressure) is generally evenly distributed. It is not uncommon for even distribution to have a tourniquet effect which can actually inhibit venous return. Moreover, present compression garments are known to slip which can cause shearing of the skin beneath.

Very few studies have quantified therapeutic pressures and there is yet to be a consensus with regard to optimal compression materials, techniques, and beneficial interface pressure metrics. There is a need for solving the above issues with current methods, and a need for developing new materials and techniques for delivering optimized therapeutic pressures for treating edema and other conditions that may benefit from compression therapy.

### SUMMARY

In one embodiment, a bandage is described. The bandage includes a primary layer, a secondary layer, and a plurality of pressure-differentiating features. The primary layer includes a continuous foam substrate and the secondary layer includes a fibrous substrate. The primary layer and the secondary layer are configured to overlay one another. The plurality of pressure-differentiating features are effective to provide localized areas of increased pressure when under compression.

In one embodiment, a compression bandage system is described. The compression bandage system includes a bandage and a compression substrate. The bandage includes a primary layer, a secondary layer, and a plurality of pressure-differentiating features. The plurality of pressure-differentiating features being effective to provide localized areas of increased pressure compared to areas without pressure-differentiating features when under compression.

In one embodiment, a method for reducing or preventing swelling in an extremity is described. The method includes wrapping at least a portion of extremity with a bandage described herein, such that the primary layer contacts a skin surface.

In one embodiment, a kit is described. The kit includes a bandage described herein and a set of instructions directing a user to wrap an extremity with the bandage.

### BRIEF DESCRIPTION OF DRAWINGS

FIG. 1a illustrates a primary layer and secondary layer adhered to one another for use in a bandage described herein.

FIG. 1b illustrates a primary layer for use in a bandage described herein.

FIG. 1c illustrates a secondary layer for use in a bandage described herein.

FIG. 2 is a cross-sectional view of a bandage described herein having pressure-differential features on the surface of the primary layer.

FIG. 3 is a cross-sectional view of a bandage described herein having pressure-differential features within the primary layer.

FIG. 4 is a cross-sectional view of a bandage described herein having pressure-differential features on the surface of the secondary layer.

FIG. 5 is a cross-sectional view of a bandage described herein having pressure-differential features within the secondary layer.

FIG. 6 is a cross-sectional view of a bandage described herein having pressure-differential features between the primary layer and the secondary layer.

FIG. 7 illustrates a compression bandage system described herein.

FIG. 8a illustrates wrapping a leg with a bandage described herein.

FIG. 8b further illustrates wrapping a leg with a bandage described herein.

FIG. 8c illustrates wrapping a leg with a compression substrate that is already wrapped with a bandage.

FIG. 8d further illustrates wrapping a leg with a compression substrate that is already wrapped with a bandage.

FIG. 8e illustrates a leg wrapped with a compression bandage system described herein.

FIG. 9 is a flow chart depicting a method for reducing or preventing swelling in an extremity.

FIG. 10 illustrates a kit including a bandage and a set of instructions for use in reducing or preventing swelling in an extremity.

### DETAILED DESCRIPTION

The present disclosure describes bandages with pressure differentials that provide for higher local tissue pressure zones next to lower local tissue pressure zones. Alternating higher and lower pressure may help to return excess of interstitial fluid into the blood capillaries more effectively compared to garments having uniformly distributed pressure. At the same time, alternating pressures may alleviate the tourniquet effect that is often experienced with current compression systems. Furthermore, the bandage design and construction (i.e., the layer of continuous foam) may reduce or eliminate skin abrasions due to slippage and skin contact with the fibrous outer layer(s). The present compression systems resemble Coban™ 2 compression systems, but with structural modifications to afford areas of increased pressure (e.g., 35-40 mmHg+10-120 mmHg). The structural modifications provide areas within the bandage of increased mass/density and these areas are pressed further into the skin surface under compression.

The bandages and compression systems of the present disclosure may be configured to extend around any area of a subject's body that is in need of compression therapy (e.g., ankle, knee, calf, thigh, entire leg, wrist, elbow, forearm, bicep, entire arm, or the like). The bandages and compression systems may be customized in size for specific areas and for specific subject identities, or may otherwise be mechanically altered to (e.g., cut) to fit. While the present disclosure focuses on treatments for swelling and lymphedema, it is readily envisioned that bandages and compression systems described herein may be used for a variety of ailments that may benefit from compression therapy (e.g., wounds, sprains, ligament injuries, or the like).

### Definitions

As used herein, “about” means ±10 percent of a given value. For example, about 10 means 9 to 11.

As used herein, “continuous” as in “continuous foam substrate” refers to a substrate without any areas that are completely void of foam. Areas void of foam could allow for skin-contact with the an outer fibrous layer which could lead to possible tissue abrasion. A continuous foam substrate increases the contact surface area between the bandage and the skin and may consequently reduce or eliminate slippage of the bandage. Slippage often contributes to tissue damage, especially when worn for a longer period of time or during activity.

As used herein, the phrase “one or more of” such as used in the phrase “one or more of A and B” or “one or more of at least one A and at least one B” means a composition may include at least one A, more than one A, at least one B, more than one B, at least one A and at least one B, more than one A and more than one B. In other words, the phrase is not intended to mean the composition must have at least one of each of A and B.

As used herein, “ankle-brachial pressure index (“ABPI”)” is the ratio of the blood pressure at the ankle to the blood pressure in the upper arm (brachium). Compared to the arm, lower blood pressure in the leg suggests blocked arteries due to peripheral artery disease (PAD). The ABPI is calculated by dividing the systolic blood pressure at the ankle by the systolic blood pressure in the arm. Depending on the ABPI, the applied pressure must be limited to avoid occlusion of the arteries, for example, with patients having peripheral artery disease. Compression must be avoided in subjects having an ABPI of <0.5. Compression should be limited to 12-25 mmHg for ABPIs between 0.5 and 0.8 (e.g. by a specifically designed compression system and/or wrapping technique). For ABPI >0.8, regular compression therapy can be applied, higher than 25 mmHg, typically around 40 mmHg in supine position.

As used herein, the phrase “stretch capacity” “elastic capacity” or “elasticity” refers to the degree to which a material can be stretched without failure (i.e., breaking).

As used herein, the phrase “stretch-recovery capacity” or “elastic recovery” refers to the degree to which a material can return to its original shape after being stretched to its elastic capacity.

As used herein, “subject” refers to a mammal, e.g., human, dog, cat, goat, horse, cow, pig, sheep, or the like.

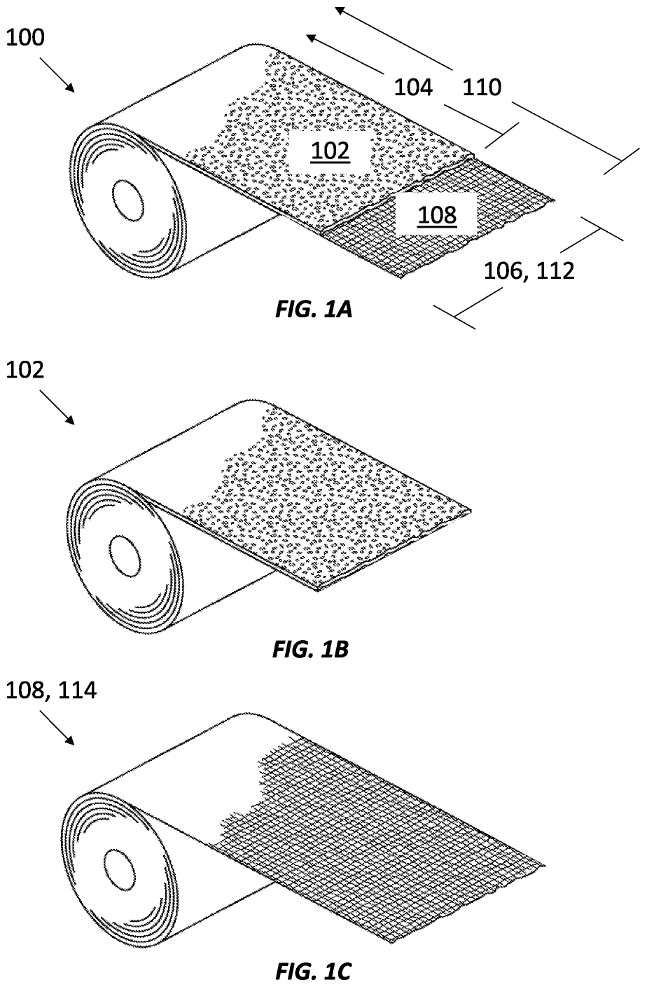

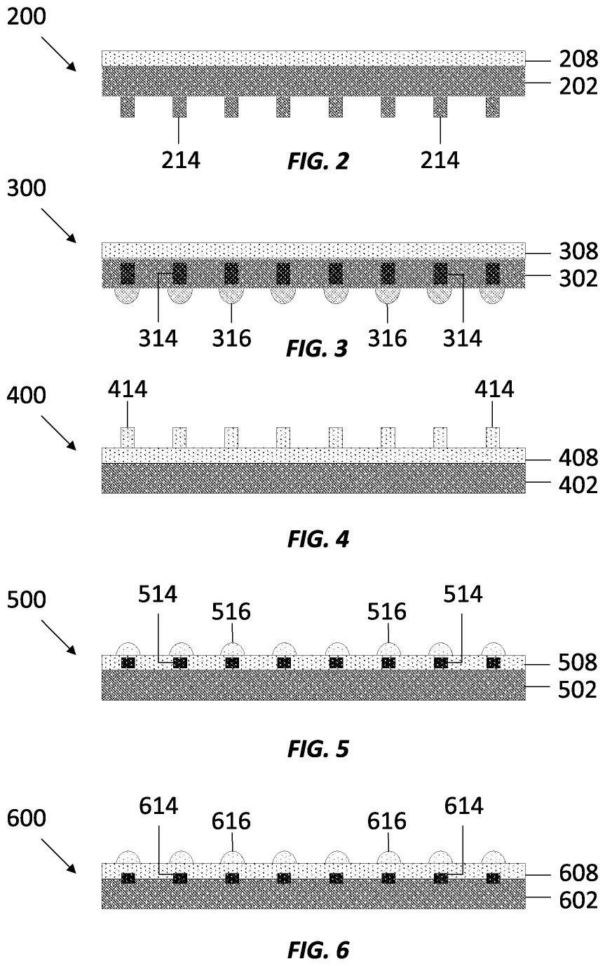

FIG. 1a depicts an exemplary bandage 100 of the present disclosure. Bandage 100 includes a primary layer 102 (shown as a continuous foam substrate) defined by a length 104 and a width 106, and a secondary layer 108 (shown as a fibrous substrate) defined by a length 110 and a width 112. As depicted, length 104<length 110; however, this is not necessary. In some applications, length 104<length 110 may assist in securing the bandage to an extremity (e.g., when the fibrous substrate is self-adhering). Likewise, it is not necessary that width 106=width 112, as depicted. In some applications, it may even be favorable for width 106<width 112 since a portion of the bandage will overlap when wrapped around an extremity (i.e., increasing the pressure in areas overlapped). Primary layer 102 and secondary layer 108 are affixed together (e.g., with an adhesive; not shown). Pressure-differentiating features are not shown, but examples of pressure-differentiating features may be seen in FIGS. 2-6.

FIG. 1b and FIG. 1c together depict an exploded view of bandage 100. FIG. 1b illustrates an exemplary primary layer 102, and FIG. 1c illustrates an exemplary secondary layer 108. Primary layer 102 and secondary layer 108 may be manufactured separately and affixed together to form bandage 100.

FIG. 2 depicts a cross-sectional view of an exemplary bandage 200. Bandage 200 includes a primary layer 202 and a secondary layer 208. A plurality of pressure differentiating features 214 are located on the surface of primary layer 202. When pressed against a skin surface, features 214 create areas of higher pressure compared to areas without said features.

FIG. 3 depicts a cross-sectional view of an exemplary bandage 300. Bandage 300 includes a primary layer 302 and a secondary layer 308. A plurality of pressure differentiating features 314 are located within primary layer 302. As shown, features 314 may create areas 316 within primary layer 302 (or secondary layer 308; not shown) of greater thickness. When pressed against a skin surface, features 314, 316 create areas of higher pressure compared to areas without said features.

FIG. 4 depicts a cross-sectional view of an exemplary bandage 400. Bandage 400 includes a primary layer 402 and a secondary layer 408. A plurality of pressure differentiating features 414 are located on the surface of secondary layer 408. When pressed against a skin surface, features 414 create areas of higher pressure compared to areas without said features.

FIG. 5 depicts a cross-sectional view of an exemplary bandage 500. Bandage 500 includes a primary layer 502 and a secondary layer 508. A plurality of pressure differentiating features 516 are located within secondary layer 508. As shown, features 514 may create areas 516 within secondary layer 508 (or primary layer 502; not shown) of greater thickness. When pressed against a skin surface, features 516 create areas of higher pressure compared to areas without said features.

FIG. 6 depicts a cross-sectional view of an exemplary bandage 600. Bandage 600 includes a primary layer 602 and a secondary layer 608. A plurality of pressure differentiating features 616 are located between primary layer 602 and secondary layer 608. Features 616 may create areas within either layer of greater thickness (not shown). When pressed against a skin surface, features 614 create areas 616 of higher pressure compared to areas without said features.

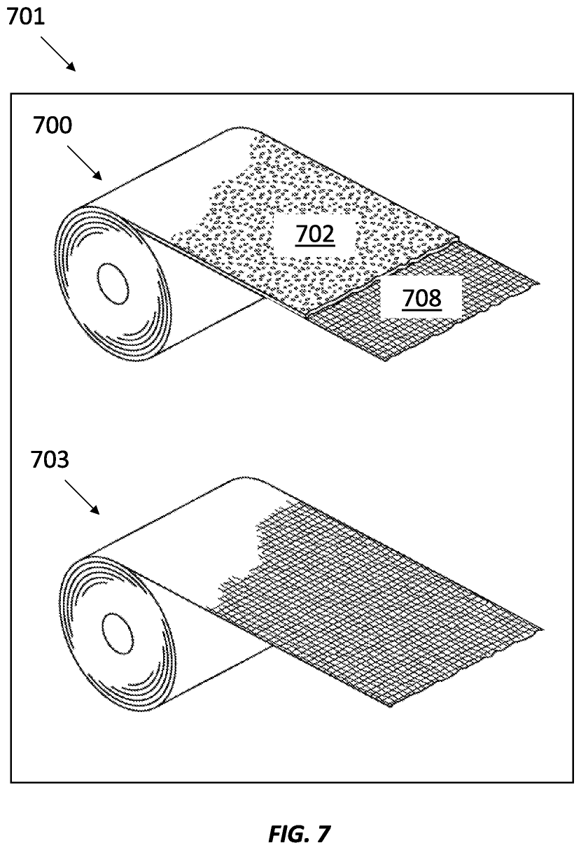

FIG. 7 depicts an exemplary compression bandage system 701. Compression bandage system 701 includes a bandage 700 and a compression substrate 703. Bandage 700 includes a primary layer 702 and a secondary layer 708. Compression substrate 703 is configured to be separately wrapped around an extremity on top of bandage 700 to provide compression pressure.

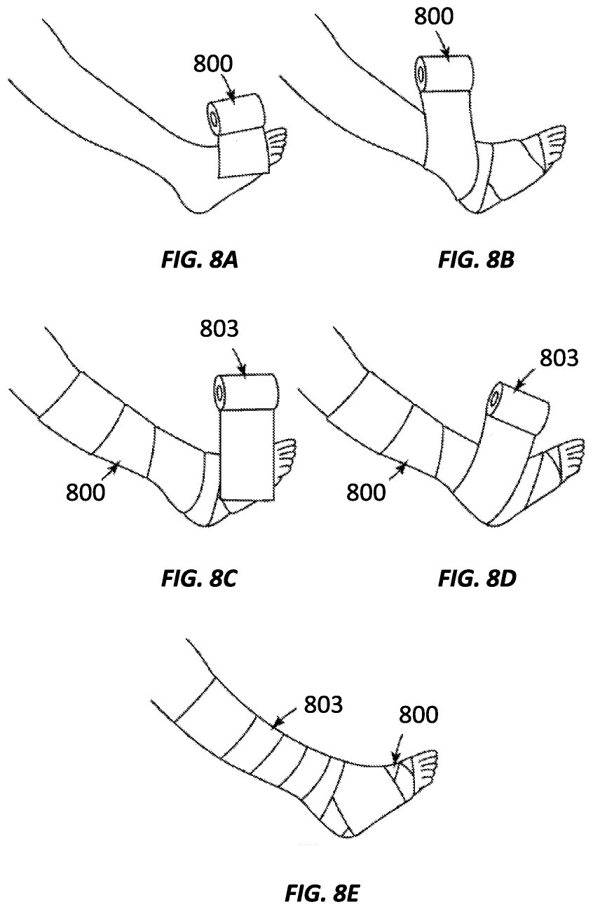

FIGS. 8a-8f are illustrations of a method for preventing or reducing swelling in a lower extremity. FIGS. 8a-b show a bandage 800 being wrapped around a foot, avoiding the heel area to prevent unfavorable wrinkling, and subsequent wrapping up the calf. It can be seen that subsequent wraps overlap a portion of the previous wrap. In some embodiments, wrapping an extremity with a bandage alone provides sufficient compression therapy. FIGS. 8c-f show a compression substrate 803 being wrapped in the same manner, but typically with a larger amount of overlap, around a lower extremity that is already wrapped with a bandage 800.

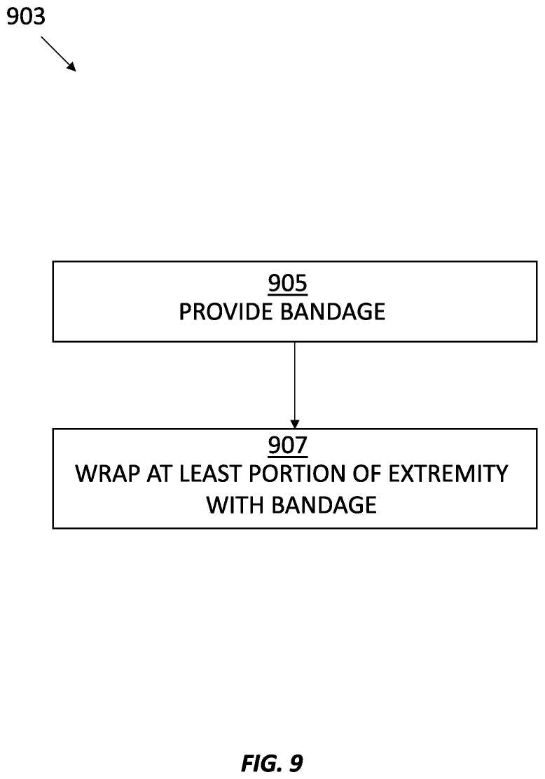

FIG. 9 is a flow chart of a method 903 for preventing or reducing swelling in an extremity. The method includes 905 providing a bandage and 907 wrapping at least a portion of an extremity with the bandage.

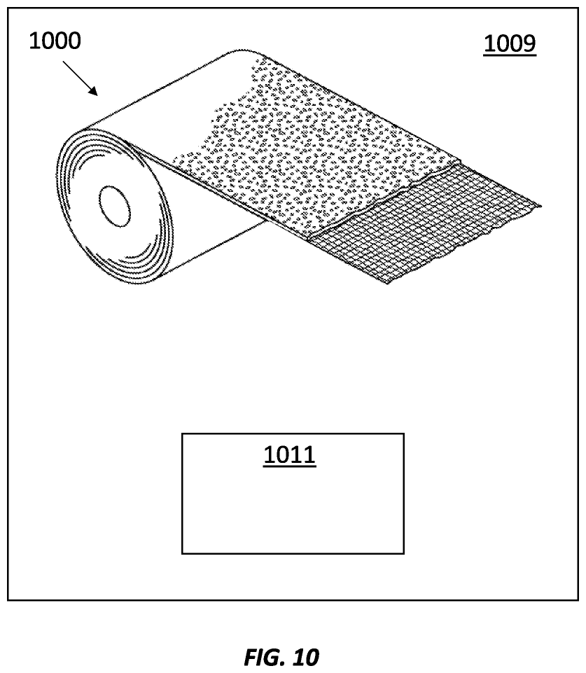

FIG. 10 depicts a kit 1009 including a bandage 1000 and a set of instructions 1011 for directing a user how to use the bandage for preventing or reducing swelling in an extremity.

### Bandages

In many embodiments, a bandage is described. The bandage may include a primary layer, a secondary layer, and a plurality of pressure-differentiating features. The primary layer may include a continuous foam substrate and the secondary layer may include a fibrous substrate. The primary layer and the secondary layer may be configured to overlay one another. The plurality of pressure-differentiating features may be effective provide localized areas of increased pressure when under compression.

The pressure differentiating features may be located anywhere within the bandage. For example, at least a portion of the pressure-differentiating features may be located on one or more surfaces of the primary layer, the secondary layer, or a combination thereof. For example, at least a portion of the pressure-differentiating features may be located between the primary layer and the secondary layer. For example, at least a portion of the pressure-differentiating features may be located within the primary layer, within the secondary layer, or a combination thereof.

Any dimension or combination of dimensions, pressure-differentiating pattern or combination of patterns described herein may be selected to accommodate size/shape of area that is to be wrapped and/or condition or severity of condition intended to be prevented or treated. For example, a bandage intended for an upper extremity may be shorter, narrower, or a combination thereof, relative to a bandage intended from a lower extremity. A bandage intended to treat an ulcer and accompanying edema may differ from a bandage intended to treat edema only. For example, a bandage may be designed to include different pressures and/or pressure patterns in locations that will overlap areas that are commonly affected by ulcers (e.g., interior ankle region).

In some embodiments, a bandage may be configured to provide a sub-bandage pressure (i.e., compression pressure) of about 1 to about 15 mmHg along areas that do not include pressure-differentiating features. For example, areas within the bandage that are void of pressure-differentiating features may include a sub-bandage pressure (in mm Hg) of about 1, 2, 3, 4, 5, 6, 7, 8, 9, 10, 11, 12, 13, 14 or 15, or a value within a range between any of the preceding values, e.g., between about 4 and about 8, between about 3 and about 6, or the like. Bandages providing a sub-bandage pressure of about 1-15 mm Hg are intended to be used with an additional compression substrate (i.e., see compression bandage system below) to provide an overall sub-bandage pressure of greater than 15 mm Hg. In some embodiments, the bandage may be configured to provide a sub-bandage (i.e., compression pressure) pressure of about 20 to about 60 mm Hg along areas that do not include pressure-differentiating features. For example, areas within the bandage that are void of pressure-differentiating features may include a sub-bandage pressure (in mm Hg) of about 20, 25, 30, 35, 40, 45, 50, 55, or 60, or a value within a range between any of the preceding values, e.g., between about 20 and about 30, between about 35 and about 50, or the like. Bandages providing a sub-bandage pressure of about 20-60 mmHg may be used as a standalone bandage (i.e., without the need for a separate compression substrate described herein.). The sub-bandage pressure may be measured according to the Sub-bandage Pressure Measurement Procedure described herein, which is based on the method reported in Melhuish et al., Phlebology, 2000, 13, 53-59. The aforementioned sub-bandage pressure values were measured with regard to a wrapped human adult ankle (22 cm ankle circumference) at a position 8 cm above the medial malleolus. In some embodiments, areas of the bandage including pressure-differentiating features may provide an increase in sub-bandage pressure of about 10 to about 120 mm Hg relative to the areas void of pressure-differentiating features. In other words, the pressure exerted on skin surfaces by the pressure-differentiating features may be about 30 mm Hg to about 180 mm Hg. For example, areas within the bandage that include pressure-differentiating features may include sub-bandage pressures (in mm Hg) of about 30, 40, 50, 60, 70, 80, 90, 100, 110, 120, 130, 140, 150, 160, 170, or 180, or a value within a range between any of the preceding values, e.g., between about 80 and about 100, between about 50 and about 120, or the like.

In some embodiments, the bandage may have an overall length of about 1 m to about 5 m in unstretched length (i.e., relaxed). The length may be chosen depending upon the type and size of an extremity intended to be wrapped. For example, the length of a bandage may be selected, in m, from about 1, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, or 5.0, or a value within a range between any of the preceding values, e.g., between about 2.4 and about 3.6, between about 4.2 and about 4.8, or the like.

In some embodiments, the bandage may have an overall width of about 40 mm to about 180 mm. The width may be chosen depending upon the type and size of an extremity intended to be wrapped. For example, the width of a bandage may be selected, in mm, from about 40, 45, 50, 55, 60, 65, 70, 75, 80, 85, 90, 95, 100, 105, 110, 115, 120, 125, 130, 135, 140, 145, 150, or 155, 160, 165, 170, 175, or 180 or a value within a range between any of the preceding values, e.g., between about 55 and about 65, between about 80 to about 100, or the like. Further, for example, a bandage intended to wrap an average-sized adult human foot and/or ankle may be about 50 mm to about 60 mm in width; a bandage intended to wrap an average-sized adult human leg below the knee may be about 100 mm to about 130 mm in width; a bandage intended to wrap an average-sized adult human leg above the knee may be about 150 mm to about 180 mm in width.

In some embodiments, the bandage may be characterized by a water vapor transition rate of at least about 240 g/m² per 24 h. A water vapor transition rate of at least about 240 g/m² per 24 h may assist in keeping the wrapped extremity comfortably dry and may reduce bacteria growth, yet allow for enough moisture to facilitate wound healing (if present).

In some embodiments, the first layer and the second layer may be adhered together. The first layer and the second layer may be affixed to one another, for example, via stitching, needle tacking, ultrasonic welding, or bonding (e.g., mechanical thermal, or chemical bonding). Adhesives for chemical bonding that are common in wound dressings include those disclosed in WO 1999/27975; WO 1999/28539; US RE 24,906; U.S. Pat. Nos. 5,849,325; and 4,871,812; the contents of each of which are included by reference herein in their entireties.

In some embodiments, the bandage may be characterized by a stretch capability (i.e., elasticity) of about 15% to about 75% in the longitudinal direction according to the Stretch Testing Procedure described herein. For example, the bandage may be characterized by a stretch capability of about 15%, 20%, 25%, 30%, 35%, 40%, 50%, 55%, 60%, 65%, or 75%, or a value within a range between any of the preceding values, e.g., between about 50% and about 60%, between about 20% and about 40%, or the like. It was observed that bandages of suitable extensibility aids in providing desirably low resting pressures and high walking pressures, as well as relative ease for users to apply the bandage at desired therapeutic pressures. Furthermore, it was found that the stretch capabilities of 15% to 75% avoided the formation of wrinkling during application. Wrinkling may during application or extended wear can lead to slippage and tissue abrasions.

In some embodiments, the bandage may be characterized by a recovery-of-stretch capability of at least 80% as determined in accordance with the Stretch Testing Procedure described herein. In some embodiments, the bandage may be characterized by a recovery-of-stretch capability of at least 90%.

In some embodiments, the bandages described herein may be used alone or in combination with an independent compression substrate.

Details of each of the bandage components are described in further detail below.

### Primary Layer

In many embodiments, the continuous foam substrate may include material(s) selected according to desired characteristics. The type(s) of foam may be selected, for example, to deliver higher or lower pressures upon compression, selected for absorption capacities, and the like. In many cases, certain features may be more suited for some conditions or may be desired based on user comfort or preference. The interaction between the continuous foam substrate and the skin provides for a friction suitable to minimize slippage and tissue abrasion.

In some embodiments, the continuous foam substrate may include open-cell foam, closed-cell foam, or a combination thereof. Closed-cell foams are more dense than open-cell foams. Accordingly, it may be expected that foam substrates comprised of closed-cell foam provide for overall higher pressures when compressed to the skin surface compared to foam substrates comprised of open-cell foam. Likewise, foam substrates having both open-cell and closed-cell foams may provide for areas of higher pressure where closed-cell foam is present. In some embodiments, the continuous foam substrate may include areas of open-cell foam and areas having closed-cell foam (or otherwise denser foam), in which areas having closed-cell foam may exert greater pressure upon the skin surface under compression, thus effectively serving as pressure-differentiating features.

Open cell foams may further allow transport of fluid and cellular debris into and within the foam. In some embodiments, the foam substrate may include open-cell foams having an average cell size of about 30 microns to about 500 microns (measured by scanning electron microscopy or light microscopy). For example, open-cell foams may have an average cell size in microns of about 30, 40, 50, 75, 100, 125, 150, 175, 200, 225, 250, 275, 300, 325, 350, 375, 400, 425, 450, 475, or 500, or a value within a range between any of the preceding values, e.g., between about 50 and about 150, between about 100 and about 250, or the like.

In some embodiments, the foam substrate may be absorbent (i.e., capable of absorbing greater than 250% aqueous saline solution when immersed for 40 min in phosphate saline containing 0.9 wt % NaCl at 37° C.). In some embodiments, the foam substrate may be non-swelling (i.e., increasing in volume by no greater than about 15% following a 30 min soak in phosphate saline containing 0.8 wt % NaCl at 37° C.). Non-swelling foams may be advantageous against skin macerations.

In some embodiments, the foam substrate may include foam materials that are hydrophilic, hydrophobic, or a combination thereof. Hydrophobic foam materials may further be treated with substances to render them more hydrophilic, substances such as surfactants (e.g., nonionic surfactants such as oxypropylene-oxyethylene block copolymers).

In some embodiments, the foam substrate may include materials selected from a polyurethane (i.e., polymers including urethane moieties, urea moieties, or a combination thereof), carboxylated butadiene-styrene, rubber, polyester, polyacrylate, polyether, polyolefin, polychloroprene, silicon, and a combination thereof. Suitable foams include such as those described in U.S. Pat. Nos. 3,908,645 and 6,548,727; the contents of each of the which are incorporated be reference herein in their entireties.

In some embodiments, the primary layer may have a width that is about 30% to about 100% of the width of the secondary layer. For example, the primary layer may have a width that is a % of the width of the secondary layer of about 30, 35, 40, 45, 50, 55, 60, 65, 70, 75, 80, 85, 90, 95, or 100, or a value within a range between any of the preceding values, e.g., between about 50 and about 75, between about 40 and about 60, or the like. In some instances, a primary layer having a width that is <100% the width of the secondary layer may prevent the doubling (or more) of pressure that could be exerted in the areas of overlap when wrapped. For example, if a primary layer has a width that is about 30% of the width of the secondary layer, then upon wrapping an extremity, the bandage may overlap about 70% of the previous wrapped portion without doubling of the foam substrate thickness. However, in other instances, overlap may provide for desired additional pressure differentiation since the areas of overlap may afford increased pressure compared to the areas that are not overlapped.

In some embodiments, the primary layer may have a width that is uniform along the length of the bandage. In other embodiments, the primary layer may have a width that is non-uniform along the length of the bandage. A non-uniform width may allow for more of less overlap of the primary layer upon wrapping an extremity. For example, more overlap of the bandage toward the distal end of the extremity and less overlap toward the proximal end of the extremity, without overlapping of the primary layer, may be accomplished by varying the width (i.e., non-uniform) of the primary layer along the length of the bandage.

In some embodiments, the primary layer may have a thickness of about 1.6 mm to about 10 mm. For example, the primary layer may have a thickness (in mm) of about 1.6, 1.8, 1.9, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, or 10 or a value within a range between any of the preceding values, e.g., between about 2 mm and about 3 mm, between about 5 mm and about 8 mm, or the like. In some embodiments, the thickness may be uniform or non-uniform along the length of the bandage. For example, one or more areas of the bandage may have a thickness greater than another area(s). Areas of greater thickness may impart greater pressures under compression. In some embodiments, a bandage having a primary layer of non-uniform thickness may allow for greater compression at the distal end of the extremity and lesser compression at the proximate end of the extremity. In other embodiments, the thickness may be uniform or non-uniform across the width along the length of the bandage. A non-uniform thickness across the width of the bandage may allow for tailoring of areas that overlap when wrapped. In other words, areas that overlap upon wrapping an extremity may have a thickness that is less than the thickness in areas that do not overlap when wrapped, or may have a thickness that is greater than the thickness in areas that do not overlap when wrapped. The difference in overall thickness may be effective to create areas of decreased or increased pressure in areas that overlap.

In some embodiments, the primary layer may include at least a portion of the pressure-differentiating features on one or more surfaces, i.e., the surface in contact with the second layer and/or the outward-facing surface (skin-facing surface) opposite the surface in contact with the second layer. For embodiments including pressure-differentiating features on a primary layer surface in contact with the second layer, it may be understood that the pressure-differentiating features are located between the first layer and the second layer. In some embodiments, the primary layer may be constructed such that the surface includes areas of greater thickness (i.e., areas of greater thickness resulting in increased pressure when under compression). Such areas of greater thickness may be constructed of the same foam material, different foam material (e.g., denser foam), or an entirely different material altogether. In some embodiments, pressure features may be installed on a surface of the primary layer by any conceivable means, e.g., molding, adhesives to adhere features or adhesives themselves, sewing, printing, stitching (e.g., stitching itself may serve as features), folding of foam, features laminated between the primary layer and secondary layer, or the like.

In some embodiments, the primary layer may include at least a portion of the pressure-differentiating features within the foam substrate. Pressure-differentiating materials may be inserted into the foam substrate or otherwise incorporated upon formation of the foam substrate.

In other embodiments, the primary layer may include only the continuous foam substrate (i.e., the primary layer is the continuous foam substrate). In yet other embodiments, the primary layer may include only the continuous foam substrate and pressure-differentiating features.

### Secondary Layer

In many embodiments, the fibrous substrate may include material(s) selected according to desired characteristics. The type(s) of fibers may be selected, for example, to deliver higher or lower pressures upon compression, selected for elasticity considerations, and the like. In many cases, certain characteristics may be more suited for some conditions or may be desired based on user comfort or preference.

In some embodiments, the fibrous substrate may be at least partly constructed from one or more mat of a woven material, a knitted material, a warp-knit material, a nonwoven material, or a combination thereof. Each mat may be constructed from longitudinally extended yarns that are elastic, inelastic, or a combination thereof. In some embodiments, more than one mat (e.g., two mats) may form the fibrous substrate. In some embodiments, a fibrous substrate having more than one mat may include elastic fibers incorporated between mats wherein the elastic fibers are oriented longitudinally.

In some embodiments, the one or more mat may be impregnated with a polymeric binder (e.g., elastomeric, nonelastomeric, or a combination thereof). In some cases, the polymeric binder may afford self-adhering properties such that the fibrous substrate will adhere to itself but not adhere to clothing, skin, hair, or the like. The polymeric binder may coat one or more major surface of the fibrous substrate and even extend throughout the thickness of the fibrous substrate. Suitable elastomeric polymeric binders may include natural rubber latex, a synthetic latex (e.g., homopolymers and copolymers of latexes of acrylics), butadienes, styrene-butadiene rubbers, chloroprenes, ethylenes (e.g., vinyl acetate/ethylene), isoprenes, nitriles, urethanes, or a combination thereof, or the like. Example elastomeric polymeric binders can be found in U.S. Pat. Nos. 3,575,782; 4,984,585; and 6,156,424, as well as in textbooks such as Neoprene Latex: Principles of Compounding and Processing, J. C. Carl, 1962, Delaware, E.I: DuPont de Nemours (see section entitled Contact Bond Adhesives, pg. 100), and Handbook of Adhesives 3rd Edition, Ed. I. Skeist, 1990, New York, Van Nostrand Reinhold (see pg. 305); the contents of each of which are incorporated by reference herein in their entireties. In other embodiments, the one or more mat is free of polymeric binder.

In some embodiments, the fibrous substrate may include materials selected from polyester, rayon, polyethyl acrylate, polyether-polyurea copolymer (e.g., Spandex), and a combination thereof.

In some embodiments, the secondary layer may have a length of about 1 m to about 5 m. For example, the length of a secondary layer may be selected, in mm, from about 1, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, or 5.0, or a value within a range between any of the preceding values, e.g., between about 2.4 and about 3.6, between about 4.2 and about 4.8, or the like.

In some embodiments, the secondary layer may have an overall width of about 40 mm to about 180 mm. The width may be chosen depending upon the type and size of an extremity intended to be wrapped. For example, the width of a secondary layer may be selected, in mm, from about 40, 45, 50, 55, 60, 65, 70, 75, 80, 85, 90, 95, 100, 105, 110, 115, 120, 125, 130, 135, 140, 145, 150, or 155, 160, 165, 170, 175, or 180 or a value within a range between any of the preceding values, e.g., between about 55 and about 65, between about 80 to about 100, or the like.

In some embodiments, the secondary layer may include at least a portion of the pressure-differentiating features on one or more surfaces, i.e., the surface in contact with the primary layer and/or the outward-facing surface opposite the surface in contact with the primary layer. In some embodiments, the secondary layer may be constructed such that a surface includes areas of greater thickness (i.e., areas of greater thickness resulting in increased pressure when under compression). Such areas of greater thickness may be constructed of the same or different fibrous material. In some embodiments, pressure features may be installed on a surface of the secondary layer by any conceivable means, e.g., molding, adhesives to adhere features or adhesives themselves, sewing, printing, stitching (e.g., stitching itself may serve as features), folding of material, features laminated between the primary layer and secondary layer, or the like.

In some embodiments, the secondary layer may include at least a portion of the pressure-differentiating within the fibrous substrate. For example, features may be woven into the fibrous substrate, fibrous substrates may be woven around features, or more than one fibrous mat may together surround features and adhere together.

In some embodiments, the secondary layer may include two fibrous mats (e.g., two nonwoven, or one nonwoven and one woven), elastic filaments between the fibrous mats, and a polymeric binder.

### Pressure-Differentiating Features

The pressure-differentiating features may be constructed of any material and may be present in any shape, configuration or pattern. The characteristics of the pressure-differentiating features may or may not be uniform along the entirety of the bandage.

In some embodiments, at least a portion of the pressure-differentiating features may be constructed from fibers, yarns, fabrics, foams, beads, polymeric hot melt adhesives, adhesives, resins, plastics (e.g., polylactic acid), silicones, or a combination thereof. For example, a fabric may have thick weft threads and thin warp yarns, where the weft thread provides the pressure-differentiating features. For example, pressure-differentiating features may be 3D printed onto or within one or more of the primary layer and the secondary layer.

In some embodiments, the pressure-differentiating features may include independent shapes, for example, spheres, hemispheres, cylinders, semicylinders, wedges, pyramids, frustra, cubes, cuboids, parallelepiped, other prisms (e.g., hexagonal prisms), cupolaes, and the like. In some embodiments, the shapes may be solid or may otherwise be hollowed. The shapes may be arranged in patterns (i.e., solid) or lattices (i.e., hollowed). For example, a honeycomb may be described as a hexagonal prism lattice.

In some embodiments, at least a portion of pressure-differentiating features may be arranged in rows. In some embodiments, the rows may be oriented parallel to the length of the bandage. In other embodiments, the rows may be oriented at an angle of 5° to 90° relative to an axis parallel to the length of the bandage The rows may be straight or non-straight (e.g., sine-type waves, zigzag-type waves). A row may be configured from one elongated feature shape, or may include a plurality of feature shapes arranged in a continuous or discontinuous line.

In some embodiments, the rows may be spaced independently at a distance selected from about 4 mm to about 20 mm. For example, the rows may be independently separated, in mm, by about 4, 5, 6, 7, 8, 9, 10, 11, 12, 13, 14, 15, 16, 17, 18, 19, or 20, or a value with a range between any of the preceding values, e.g., between about 5 and about 10, between about 6 and about 12, or the like. In some embodiments, the rows may be separated by a uniform or non-uniform distance along the bandage. In some instances, it may be beneficial for a bandage to include rows closer together at an end intended to wrap a distal portion of an extremity and rows further apart at an end intended to wrap a proximal portion of the extremity.

In some embodiments, at least a portion of the pressure-differentiating features may be arranged in a lattice. Example lattices may include cross-hatch, brick, diamond, honeycomb, square or hexagonal circle packing, or the like.

In some embodiments, the difference in pressure across the pressure-differentiating features may be relatively uniform (i.e., ±5% mm Hg) along the length of the bandage. In other embodiments, the difference in pressure across the pressure-differentiating features may be non-uniform (i.e., >5% mm Hg difference) along the length of the bandage. Non-uniform pressure-differentials may be achieved, for example, by including different structural features, different spacing of features, different patterns of features, absence of features, or the like. In some embodiments, it may be beneficial to include certain features configured to overlay certain areas of an extremity. For example, the knee and the elbow may experience greater pressures with activity and therefore may benefit in having different pressure features in said areas of the bandage. Likewise, it may be beneficial to include different pressure features along areas that are commonly afflicted with wounds, e.g., ankle ulcers.

In some embodiments, the pressure-differentiating features may extend over a portion of the length of the bandage. For example, the pressure-differentiating features may extend over about 20% to 100% of the length of the bandage. In some embodiments, the pressure-differentiating features may extend over a percentage of the length of the bandage of about 20, 25, 30, 35, 40, 45, 50, 55, 60, 65, 70, 75, 80, 85, 90, 95, or 100, or a value within a range between any of the preceding values, e.g., between about 75 and about 90, between about 50 and about 80, or the like.

In some embodiments, the pressure-differentiating features may extend over a portion of the width of the bandage. For example, the pressure-differentiating features may extend over about 10% to about 100% of the width of the bandage. In some embodiments, the pressure-differentiating features may extend over a percentage of the width of the bandage of about 10, 15, 20, 25, 30, 35, 40, 45, 50, 55, 60, 65, 70, 75, 80, 85, 90, 95, or 100, or a value within a range between any of the preceding values, e.g., between about 30 and about 50, between about 20 and about 70, or the like. In some embodiments, the pressure-differentiating features may extend over no more than 50% of the width of the bandage. Pressure-differentiating features extending over a percentage of less than 100% of the width of the bandage may allow for overlapping of the bandage upon application to an extremity without overlapping of areas with pressure-differentiating features.

### Compression Bandage Systems

In various embodiments, a compression bandage system is described. The compression bandage system may include a bandage described herein and a compression substrate. The compression bandage system is intended to be applied to subject wherein the bandage is first wrapped around an extremity followed by wrapping of the extremity with the compression substrate.

All compression substrates described herein are elastic. In many embodiments, the compression substrate may be constructed from any of the materials described above regarding the fibrous substrate (i.e., within the bandage), provided that the compression substrate include elastic fibers. Likewise, the compression substrate may be characterized by a stretch capability (elasticity) similar to or equivalent to the elasticity values described above regarding the fibrous substrate within the bandage. In some embodiments, the fibrous substrate and the compression substrate may be identical or non-identical.

In some embodiments, the compression substrate may be characterized by a stretch capability (i.e., elasticity) of about 15% to about 75% in the longitudinal direction according to the Stretch Testing Procedure described herein. For example, the compression substrate may be characterized by a stretch capability of about 15%, 20%, 25%, 30%, 35%, 40%, 50%, 55%, 60%, 65%, or 75%, or a value within a range between any of the preceding values, e.g., between about 50% and about 60%, between about 20% and about 40%, or the like.

In some embodiments, the compression substrate may be characterized by a recovery-of-stretch capability of at least 80% as determined in accordance with the Stretch Testing Procedure described herein. In some embodiments, the compression substrate may be characterized by a recovery-of-stretch capability of at least 90%.

In many embodiments, the compression substrate may be configured to provide a sub-bandage (i.e., compression) pressure of about 5 to about 60 mm Hg. For example, the compression substrate may deliver a sub-bandage pressure (in mm Hg) of about 5, 10, 15, 20, 25, 30, 35, 40, 45, 50, 55, 60, or a value within a range between any of the preceding values, e.g., between about 25 and about 35, between about 30 and about 50, or the like. The sub-bandage pressure may be measured according to the Sub-bandage Pressure Measurement Procedure described herein, which is based on the method reported in Melhuish et al., Phlebology, 2000, 13, 53-59. The aforementioned sub-bandage pressure values were measured with regard to a wrapped human adult ankle (22 cm ankle circumference) at a position 8 cm above the medial malleolus.

In some embodiments, the bandage and the compression substrate are configured to adhere to one another under elastic extension without the use of a fastening mechanism, e.g., with use of polymeric binders in one or more of the secondary layer within the bandage and the compression substrate. Self-adhering between the bandage and the compression substrate may further minimize or eliminate slippage and/or wrinkling between the components during application and extended wear.

### Methods for Reducing or Preventing Swelling

In many embodiments, a method for reducing or preventing swelling in an extremity is described. The method may include wrapping (i.e., spirally winding) at least a portion of extremity with a bandage described herein. The bandage is to be applied with the primary layer (i.e., foam substrate) facing the surface of the extremity.

In many embodiments, the bandage is applied first to a distal area of the extremity and wrapped upward toward a proximal area of the extremity.

In some embodiments, the wrapping may involve wrapping the extremity with equal pressure along the length of the extremity. In other embodiments, the wrapping may involve wrapping a most distal point of the extremity such that said area experiences greater pressure and a most proximal point of the extremity experiences the least pressure.

In many embodiments, the method may further include wrapping (i.e., spirally winding) at least a portion of extremity with a compression bandage described herein. The compression bandage intended to be wrapped over the first bandage.

In some embodiments, the wrapping may involve successive wrapping layers that independently overlay about 30% to about 70% of the width of a previous wrapping layer. For example, a successive wrapping layer may overlay a percentage of the width of a previous wrapping layer of about 30, 35, 40, 45, 50, 55, 60, 65, or 70, or a value within a range between any of the preceding values, e.g., between about 30 and about 35, between about 40 and about 60, or the like.

In some embodiments, the method may further include measuring the Ankle-Brachial Pressure Index (ABPI) of the subject and selecting an appropriate bandage or compression bandage system based on the ABPI value.

In some embodiments, the bandage or the compression bandage system may be used to treat pitting edema (i.e., type of swelling). For example, the edema may be a symptom of a condition involving heart valve problems, low protein levels, deep venous thrombosis, lung disease, congestive heart failure, venous insufficiency, liver disease, kidney failure, obesity, pregnancy, intravenous administration of fluids, medication side effects, surgical side effects, or the like.

In some embodiments, the bandage or the compression bandage system may be used to treat non-pitting edema (i.e., type of swelling). For example, the edema may be a symptom of lymphedema.

### Kits

In many embodiments, a kit is described. The kit may include a bandage described herein and a set of instructions directing a user to wrap an extremity with the bandage according to any of the method steps for reducing or preventing swelling (e.g., edema) described herein.

In many embodiments, the kit may further include a compression substrate described herein.

### Methods of Preparing Bandages

In various embodiments, a method of preparing a bandage described herein is described. The method may include providing one or more of the fibrous substrate and the continuous foam substrate, and modifying either the fibrous substrate, the continuous foam substrate, or both, to include areas of greater thickness (e.g., folds, added or removed substrate material, added exogenous materials (e.g., hot-melts, beads, or the like), or a combination thereof).

In various embodiments, a method of preparing at least a portion of a bandage described herein is described. The method may include providing a continuous foam substrate, folding portions of the continuous foam substrate to provide folded segments (e.g., in any pattern described herein), and adhering the folded segments to form at least a portion of the plurality of pressure-differentiating features. In some embodiments, the method may further include providing a fibrous substrate and adhering the fibrous substrate and the continuous foam substrate. The fibrous substrate may be adhered to the continuous foam substrate before or after the folding of the continuous foam substrate. In many embodiments, the continuous foam substrate is adhered to the fibrous substrate prior to folding segments of the continuous foam substrate.

In some embodiments, folding portions of the continuous foam substrate may form loop shapes within the continuous foam substrate. For example, loop shapes may be formed by pinching together areas of the continuous foam substrate. For embodiments including an affixed self-adhering fibrous substrate, the fibrous substrate may serve to adhere the folding segments within the folded loop shapes, i.e., the fibrous substrate of one area may contact another area upon folding thereby securing the fold. In other embodiments, adhering the folded segments may include application of adhesive, stitching, or the like.

### Examples

Objects and advantages of this disclosure are further illustrated by the following examples, but the particular materials and amounts thereof recited in these examples, as well as other conditions and details, should not be construed to unduly limit this disclosure. These examples are merely for illustrative purposes only and are not meant to be limiting on the scope of the appended claims.

### Test Methods

### Elasticity (Stretch) and Elastic (Stretch) Recovery Testing Procedure

A stretch testing instrument consisting of the following was used: A frame with a fixed clamp at the top (upper clamp); a separate clamp (or other means) for attaching a weight to the bottom of the test specimen (lower clamp); a scale to measure the span of bench marks on the specimen graduated in units of 1 mm ( 1/24 inch) (or in units of percent of original gage length)±0.1% and a weight with an attached hook and a mass of 1000 g. The total tensioning weight including the weight having a mass of 1000 g and the lower clamp weighing 45 g was 1045 g.

Test specimen was allowed to condition at 23° C. and 50% relative humidity for 24 hours. Testing was performed at 23° C. and 50% relative humidity.

After conditioning, the test specimen was placed on a smooth flat surface and allowed to relax for at least 2 minutes. Thereafter a test sample dimension of 50.8 by 304.8 mm (2 by 12 inches) with the long direction parallel to test direction was prepared, e.g., by punching using a cutting template. (In particular, a template consisting of protruding cutting knives embedded in a wooden housing of about 290 mm×90 mm×17 mm dimension was used for cutting the test specimens to the required test sample dimension of 50.8 mm×304.8 mm. The template was placed in alignment on top of a conditioned, fully relaxed test specimen with the protruding knives facing the test specimen, and the test sample was cut by applying pressure (typically manually using a hammer) against the protruding knives opposing smooth wooden surface.)

After allowing the test sample to relax for at least 2 minutes, two bench marks of 127±1 mm (5 inch) apart are placed from the center of the test sample.

The two transverse ends of the test sample are then folded over until their outer edges are in line with the two bench marks. (By doing this, the ends of the test sample are reinforced and the long dimension was reduced from 304.8 mm (12 inches) down to 215.9 mm (8.5 inches) in length.)

One (transverse) end of the test specimen was then clamped with the upper clamp to the frame such that the test sample hangs freely and such that the lower edge of the clamp was in exact alignment with the upper bench mark.

On the lower (transverse) end of the test sample and in vertical alignment to the upper clamp, the lower clamp was attached, such that the upper edge of the clamp was in exact alignment with the lower bench mark. Accordingly the original test length (OL) was the distance between the two bench marks, 127 mm. Directly thereafter, the attachment hook of the 1000 g weight was inserted in an opening of the lower clamp, and then slowly (over approximately 5 s) the weight was allowed to hang freely thereby exerting tension to the test sample.

Using the scale, the distance between the bench marks was measured to the nearest 0.5 mm (or to the nearest 1% of original gage length), after the freely suspending the weight for 60±2 sec. The measured distance was the stretch length SL.

Directly thereafter, the weight and lower clamp are removed and the test sample was allowed to recover without tension.

Using the scale, the distance between the bench marks was measured to the nearest 0.5 mm (or to the nearest 1% of original gage length), after the tension has been removed for 120±4 sec. The measured distance was the recovery length RL.

Percent Stretch and Percent Recovery-of-Stretch are calculated as follows:

$$\% Stretch = 100 \times ( SL - OL ) / OL$$

$$\% Recovery - of - Stretch = 100 \times ( SL - RL ) / ( SL - OL )$$

6 test samples are tested for each test specimen and the mean values for % stretch and % recovery-of-stretch are calculated.

### Water Vapor Transmission Rate (WVTR)

WVTR was measured in accordance with ASTM E 398-03 using a Water Permeability Tester L80-5000 (available from Lyssy AG, 8702 Zollikon, Switzerland), wherein the conditions of the wet (high humidity) chamber are 37.8° C. and 100% relative humidity and the conditions of the dry (low humidity) chamber are 37.8° C. and 10% relative humidity.

For measurements of bandage samples, the foam layer faced the wet (high humidity) chamber while then the compression substrate was facing the opposing dry (low humidity) chamber.

If a material to be tested has any bias from one face to the other face, the face of the material that was to be facing towards the skin of the patient should be oriented towards the wet chamber.

### Saline Absorbency Test

A dry 5.1 cm by 5.1 cm sample (2 inch by 2 inch) was weighed to obtain dry weight (DW).

Directly thereafter, the sample was immersed in phosphate-buffered saline (Sigma-Aldrich Chemical Co., Milwaukee, Wisconsin; dry powder blend dissolved in water to 0.9% NaCl) for thirty minutes at 37° C.

Upon removal, the sample was allowed to drip freely for thirty seconds, and re-weighed to obtain wet weight (WW).

Percent absorbency of the sample was determined using the formula:

$$\% Absorbency = ( ( WW - DW ) / DW ) ) \times 100.$$

Three replications are performed with the reported result being the mean value.

### Saline Swelling Test

The width (dW), length (dL), and thickness (dT) of an approximate 5.1 cm by 5.1 cm (2 inches by 2 inches) dry sample are measured using a scale graduated in units of 0.5 mm ( 1/48 inch).

Directly thereafter, the sample was immersed in phosphate-buffered saline (Sigma-Aldrich Chemical Co., Milwaukee, Wisconsin; dry powder blend dissolved in water to 0.9% NaCl) for thirty minutes at 37° C.

Upon removal, the sample was allowed to drip freely for thirty seconds, and all three dimensions of the sample were immediately re-measured to obtain “wet” width (wW), length (wL) and thickness (wT).

Percent swelling of the sample was determined using the formula:

$$\% Swelling = [ ( ( wW * wL * wT ) ) - ( dW * d L * dT ) ) / ( dW * d L * dT ) ] \times 100.$$

Three replications are performed with the reported result being the mean value.

### Sub-Bandage Pressure Measurement

Sub-bandage pressure was monitored using three pressure transducers taped to the skin of a leg of a healthy person (at rest and walking) under the applied compression bandage system. The procedure was based on the procedure described by Melhuish et al, in “Evaluation of Compression under an Elastic Tubular Bandage Utilized as an Introduction to Compression Therapy in the Treatment of Venous Leg Ulcers” Phlebology (2000) 15:53-59.

Three strain gauge temperature-compensated (15-40° C.) pressure transducers, each having a diameter of 13 mm and a thickness of 3 mm (available by Gaeltec Ltd., Dunvegan, Isle of Skye, Scotland IV55 8GU) are used.

Before measurement, instrumentation was allowed a settling time of at least 5 minutes, and the pressure transducers are calibrated in an air chamber against a sphygmomanometer (available by Gaeltec Ltd., Dunvegan, Isle of Skye, Scotland IV55 8GU).

The transducers (typically connected via amplifiers and filters to a computer for data storage and analysis) are then placed on the lateral aspect of the non-dominant leg (of the test person) in line with the medial malleolus positioned as follows:
Sensor 1: at 8 cm above the lateral malleolus (i.e. approximately at the origin of the Achilles tendon);
Sensor 3: at 5 cm below the caput fibulae; and
Sensor 2: at a position located exactly at the midpoint between the positions Sensors 1 and 3.

The sensors are fixed into positioned by taping down their connecting wires with one or two small pieces of tape; the transducer itself was not covered with tape.

The compression bandage system was applied (typically by an experienced nurse).

For measurement of sub-bandage resting pressures-pressure measurements are taken over a 5-minute period (typically with 20 measurement readings per second) with the test person sitting in an upright position with his legs extending horizontally and being supported from underneath. From this measurement set, a selection of a set of consecutive, steady (i.e., no statistically significant step changes in measured pressure readings) pressure readings over a period of at least one minute was made, and the mean value of said pressure readings was determined and reported in mm Hg. (The aforesaid selection was made to exclude significant changes in pressure readings resulting e.g., from movement of the test person. If no set of consecutive, relatively steady pressure readings over a period of at least one minute was observed, the measurement was repeated.)

For measurement of sub-bandage walking pressures-pressure measurements are taken over a 5-minute period (typically with 20 measurement readings per second) with the test person walking on a treadmill at no incline and at a velocity of 2.5 km/h. From this measurement set, a selection of a set of consecutive, steady (i.e., no statistically significant step changes in measured pressure readings) pressure readings over a period of at least one minute was made, and the mean value of said pressure readings was determined and reported in mm Hg. (The aforesaid selection was made to exclude statistically significant step changes in pressure measurement resulting e.g., from changes in walking speed, stumbling, etc. of the test person. If no set of consecutive, steady pressure readings over a period of at least one minute was observed, the measurement was repeated.)

### Slippage Measurement

After a compression bandage system was applied to the leg of a healthy test person, a marker line was placed directly above the upper edge of the applied compression bandage system. Downward slippage (DS) was then determined, as the difference (measured by means of a scale graduated in units of 1 mm (±0.1%)) between the height of the upper edge of initially applied bandage system (initial height IH) and the height of the upper edge of the applied compression bandage system after 24 h of wear (height afterwards HA), DS=IH−HA. Results are reported in cm.

The following examples regarding bandages and compression bandage systems are without pressure-differentiating features. Pressure-differentiating features may be added to the following systems during or after manufacturing of each component and tested accordingly. The following bandages and compression bandage systems without pressure-differentiating features were first disclosed in EP 2322124, the contents of which are incorporated by reference herein in its entirety.

### Preparation of Bandages

A fibrous substrate (secondary layer), in each case, a self-adherent elastomeric web material (having dimensions of 10 cm by 2 m) that does not adhere to clothing, hair or skin was used. Similar to the compression substrate, the sheet material for the fibrous substrate was prepared according to the process described in U.S. Pat. No. 4,984,584 using 280 denier Spandex yarns at 10 epi between two nonwoven webs as described above with a draw ratio of about 3.5:1 and impregnating (coating weight of 52 g/m²) with a latex based fluid binder mixture as described above. After drying, the resulting sheet material (having a relaxed basis weight of 179 g/m²) was slit into strips of 10 cm wide and 2 meters long (unstretched dimensions) for use as elastic substrate. The fibrous substrate showed a stretch capability in the longitudinal direction of 110% and a recovery-of-stretch capability in the longitudinal direction of 98%; and was referred to in the following as ES.

Four different polyurethane foams were used for the continuous foam substrate (primary layer) as follows: A hydrophobic polyurethane foam available from THE WOODBRIDGE GROUP (Mississauga, Ontario, Canada) under the trade designation BIOFREE SM 25; referred to in the following as F1. F1 had a thickness of 4 mm, a measured saline absorbency of 2969% and a saline swelling of 0.4%.

A hydrophobic polyurethane foam having hydrophilic characteristics available from Fulflex, Inc (Middelton, R.I., USA) under trade designation POLYCRIL 400; referred to in the following as F2. F2 had a thickness of 4 mm, a measured saline absorbency of 1089% and a saline swelling of 8%.

A hydrophilic polyurethane foam available from Fulflex, Inc (Middelton, R.I., USA) under trade designation POLYCRIL 300; referred to in the following as F3. F3 had a thickness of 3 mm, a measured saline absorbency of 560% and a saline swelling of 8%.

A hydrophilic polyurethane foam available from Corpura B.V. (Etten-Leur, Netherlands) under the trade designation VIVO MFC.03; referred to in the following as F4. F4 had a thickness of 3 mm, a measured saline absorbency of 1805% and a saline swelling of 26%.

In manually preparing the foam-elastic substrate laminates, acrylic adhesive based transfer adhesive strips (thickness 76.2 μm, width of 16 and/or 28 cm) were applied extending lengthwise across the width (starting from the two longitudinal edges working towards the center) of a face of elongated foam layer sheet having outer dimensions of 31 to 49 cm by 2 to 2.5 m for F1 to F4. Slight pressure was applied by hand, establishing a first contact between adhesive and foam, and to further enhance good contact, the transfer adhesive was then manually rolled-over with a rubber hand roller over its release liner, rolling two to three times over the entire length and width of the foam-adhesive composite. After removing the release liner of the foam-adhesive composite, a face of a relaxed fibrous substrate (dimensions 10 cm by 2 m) was placed over the exposed face of the transfer adhesive. Depending on the particular width of the foam layer sheet, this step was repeated with two or three additional relaxed elastic substrates. (Previous to step of application to the adhesive, each elastic substrate was placed flat on a smooth surface and allowed to relax completely for at least 2 minutes.) To ensure good contact between the fibrous substrate(s) and the adhesive, the composite was turned over and the foam side was manually rolled-over with a rubber hand roller. The resulting composite was cut to width and in length by removing around 5 mm at both transverse ends (both relative to the width and length of the elastic substrate(s)) to yield inner bandages having relaxed dimensions of 10 cm and around 1.9 m, with the foam layer being co-extensive with the elastic substrate. The bandages were then wound on cores having an inner diameter of 30 mm for later use as inner bandage.

The following table summarized the prepared elastic substrate/foam laminates.

| Designation | WVTR (g/m²/24 h) | % Stretch* | % Recovery* |
|---|---|---|---|
| ES-F1 | 740 | 39 | 97 |
| ES-F2 | 799 | 45 | 96 |
| ES-F3 | 401 | 38 | 96 |
| ES-F4 | 806 | 69 | 98 |

*in the longitudinal direction

### Preparation of Compression Substrates

Self-adhering compression substrates that do not adhere to clothing, hair or skin were prepared according to the process described in U.S. Pat. No. 4,984,584, the contents of which are incorporated by reference herein in its entirety. In particular two thin dry-laid acrylic-binder-bonded nonwoven PET (1.5 denier) fibrous webs, each having a basis weight of 11 g/m², were brought into contact with 560 denier Spandex elastic yarns, longitudinally aligned and spaced apart (either 10, 14 or 20 epi) and partially extended (the draw ratio (ratio of stretched length to relaxed length of yarn) being selected to be either about 1.5:1 or 1.75:1) to provide a composite construction with the yarns between the two nonwoven webs, which was subsequently impregnated (coating weights between 31 and 52 g/m²) with a latex based fluid binder mixture including 69% (based on fluid weight) of an aqueous anionic colloidal dispersion of 2,3 dichloro-1,3-butadiene and chloroprene copolymer (about 50% solids, 40% chlorine, Brookfield viscosity (spindle 1, 6 &30 rpm) at 25° C. 10 cps) and 31% (based on fluid weight) of an aqueous dispersion of aromatic modified hydrocarbon resin having a ring and ball softening point of about 70° C. (about 55% solids, Brookfield viscosity at 25° C. 1,000 cps) plus relative to 100 parts of copolymer 4 parts of zinc oxide, 2 parts antioxidant, 0.5 parts pigment and 0.16 parts of a defoamer. After drying, the resulting sheet material was slit into bandages 10 cm wide and between 2 to 2.5 meters long (unstretched dimensions).

The following table summarized the prepared self-adhering compression substrate for use as outer bandages:

TABLE 2

| | | | Coating | Relaxed | | | |
|---|---|---|---|---|---|---|---|
| | | Draw | weight | basis weight | | | WVTR |
| Designation | epi | ratio | (g/m²) | (g/m²) | % Stretch | % Recovery* | (g/m²/24 h) |
| O1 | 10 | ~1.5:4 | 31 | 130 | 50 | 98 | 3470 |
| O2 | 14 | ~1.5:3 | 45 | 140 | 37 | 98 | 3608 |
| O3 | 14 | ~1.75:1 | 52 | 162 | 52 | 97 | — |
| O4 | 20 | ~1.75:1 | 45 | 173 | 55 | 98 | — |

*in the longitudinal direction

### Compression Bandage Systems

Exemplary bandages (i.e., inner bandage) (Table 1) in conjunction with compression substrates (i.e., outer bandage) (Table 2) as listed in the following table were used in testing (summarized below) on legs of healthy, in house volunteers.

TABLE 3

| Example No. | Outer Bandage | Inner Bandage |
|---|---|---|
| 1 | O1 | ES-F2 |
| 2 | 02 | ES-F2 |
| 3 | 03 | ES-F2 |
| 4 | 04 | ES-F2 |
| 5 | 02 | ES-F1 |
| 6 | 02 | ES-F3 |
| 7 | 02 | ES-F4 |
| C1 | 4 layer compression wrap | |

Also a four layer compression bandage commercially available under the trade designation PROFORE (18-25 cm ankle pack) from Smith & Nephew Medical Ltd. (Hull HU3 2BN, England) was used as a comparative example, C1. This bandage consists of an inner layer of orthopedic wool (a polyester-based “wool”), a second layer crepe bandage, a third layer of light compression bandage and a fourth layer of self-adherent flexible bandage.

### Pressure-Differentiating Features

### PLA Printed Pressure-Differentiating Features

A 10 cm by 25 cm piece of the foam substrate, taken from the comfort layer of the Coban™ 2 Two-Layer Compression System (3M™; Maplewood, MN), was used as substrate for 3D printing with a N2 Plus 3D printer from Raise3D. Lines of polylactic acid (9 cm long, 1.3 mm wide, approx. 1 mm thick; PLA black, Ultrafuse from BASF; Ludwigshafen, DE) were printed onto the foam substrate across the width and parallel to the end, symmetrically (i.e., leaving 5 mm space along the substrate edge on both sides). The amount of printed material was 0.1 g per line, which consisted of four individual layers, and the resin was heated to a temperature of 210° C. After printing, the foam layer was laminated with a fibrous substrate, taken from Coban™ 2 Two-Layer Compression System (3M™; Maplewood, MN), using a two-roller laminating station comprising of a steel and a rubber roll of 8 cm diameter each and using a lamination pressure of 4 bar and a lamination speed of 16 mm/see such that the pressure-differentiating features were situated between the foam substrate and the fibrous substrate.

Another example of the same dimensions was made by directly printing the PLA features as described above directly on the foam surface of a comfort layer of the of Coban™ 2 Two-Layer Compression System.

### Pressure-Differentiating Features Realized by Folding the Complete Layer

A 45 cm long and 10 cm wide piece of the comfort layer (layer 1) of the Coban™ 2 Two-Layer Compression System (3M™; Maplewood, MN), was modified by introducing equidistant folds. The folds were introduced by the following procedure: 20 cuboids of the dimension 140 mm×10 mm×5 mm were placed parallelly along the long side on a table so that the areas of 140 mm×10 cm were facing to the table and to the top. The distance between the first two cuboids was approximately 5 mm. The comfort layer was laid above the cuboids with the foam facing to the top (away from the cuboids) while leaving 2 cm length of the cuboids uncovered on each side. One end of the layer was fixed with tape to the table next to the first cuboid. Then, a 20 cm long and 1 mm thick blade was used to plug the foam into the space, touching the table, between the cuboids. Then, the blade was removed and the neighbored cuboid was moved closely to the first cuboid. A strong pressure was applied manually to press the neighbored cuboids together so that the Coban-like surfaces facing each other stick to each other. This procedure was repeated with the residual cuboids. Finally, the sample is removed from the cuboids. The folds remain.

### Measuring the Effect of the Pressure-Differentiating Features (Method A)

Modeling clay may be applied and evenly distributed on a 15 cm wide and 25 cm long wide cotton sheet in a press. The resulting clay thickness is adjusted to 3 mm thickness by distance holders. The clay sheet is wrapped around a rigid cylinder made of polymethylmethacrylate (PMMA) with a radius of 4 cm and 30 cm length. The sheet is fixed with a PVC backing adhesive tape (3M 471) at the shorter ends. The experimental substrate is laid on the clay surface with the foam facing to the clay. The ends of the sheet are fixed with the PVC adhesive tape to the cylinder. The cylinder is then placed on a winding machine and a long piece of 10 cm wide compression bandage of Coban 2 Two-Layer Compression System is fixed at one shorter end with the PVC tape at the fixation location of the other elements on the cylinder. The compression bandage is then wound around the cylinder for two full turns with a tension of 13 N/10 cm and a speed of one turn per 5 seconds. After one minute, the compression layer is removed again. The resulting profile of the clay layer may be visually assessed.

### Measuring the Effect of the Pressure-Differentiating Features (Method B)

A pressure mapping sensor and system software (I-Scan from TekScan, Inc. South Boston, MA, USA; sensor 5101; sensor area of 112 mm×112 mm) may be used to measure the pressure distribution created by the applied compression bandage system. The mapping sensor is fixed on a hollow, rigid cylinder made of polymethylmethacrylate (PMMA) with a radius of 4 cm and 30 cm length and was fixed with 3M PVC backing adhesive tape (3M 471) at the edges. The experimental substrate is laid above the sensor. The ends of the substrate are fixed with PVC adhesive tape to the cylinder. The cylinder is then placed on a winding machine and a long piece of 10 cm wide compression bandage of Coban 2 Two-Layer Compression System was fixed at one shorter end with the PVC tape to the cylinder at the area, which is not covered with the sensor map. The compression bandage is then wound around the cylinder for two full turns with a tension of 13 N/10 cm and a speed of one turn per 5 seconds. The sensor is then connected to the reading unit (USB-Evolution-Handle) and the pressure distribution map is read out and compared with the one obtained with from the same setup but using Coban 2 Two-Layer Compression System without modification (i.e., without pressure-differentiating features).

Zones with the pressure-differentiating features will result in significantly higher pressure readings compared to the neighbored areas without pressure-differentiating features. The same technique will show higher local pressures can be achieved than with Coban 2 comfort layer samples without pressure-differentiating features.

### Method of Applying Compression Bandage Systems

The bandage with foam layer facing towards and contacting the skin was wound onto the lower leg without any tension in a simple (ascending) spiral technique using about a 10% overlap, starting at the base of the toes and ending just below the fibular head. After cutting any excess bandage length, the applied inner bandage was then temporarily secured at the end of the last wrap with a small piece of medical tape, such as 3M MICROPORE (available by 3M Company, St. Paul, USA). Subsequently, the outer bandage was applied at full stretch starting at the base of the toes using two spiral turns to secure the bandage and then two figure-of-eight turns around the ankle joint (to ensure the heel was completely covered). Application was continued then in a simple (ascending) spiral technique with a 50% overlap ending just below the fibular head and if necessary cutting off any excess length of the bandage.

### Sub-Bandage Pressure Testing (1)

Four sets of 12 in-house, healthy volunteers were used for four sets of comparative testing of sub-bandage pressure. Compression bandaging systems of Examples 1 to 4 were each compared to comparative C1. In all testing—measuring pressures at rest and during walking in accordance to the sub-bandage pressure measurement procedure detailed above—the compression bandage system was applied to the non-dominant leg of the volunteer. The same volunteer was used with the testing of one of Examples 1 to 4 and in each case C1 in sequence. After testing one system, it was removed, leaving the three pressure transducers untouched in their secured positions. The pressure transducers were re-zeroed appropriately. The compression substrate was applied and pressure measurements were made. The means for the sub-bandage pressure measurement results for all 12 volunteers were computed and are reported in Table 4.

TABLE 4

| Example | Sensor 1 | Sensor 2 | Sensor 3 | Sensor 1 | Sensor 2 | Sensor 3 |
|---|---|---|---|---|---|---|
| No. | Rest (mmHg) | Rest (mmHg) | Rest (mmHg) | Walk (mmHg) | Walk (mmHg) | Walk (mmHg) |
| 1 | 67.00 | 31.75 | 32.00 | 65.50 | 37.25 | 33.50 |
| C1 | 57.75 | 36.50 | 35.75 | 59.00 | 40.25 | 35.00 |
| 2 | 67.75 | 41.00 | 37.50 | 64.25 | 37.00 | 36.50 |
| C1 | 60.00 | 37.50 | 36.50 | 52.50 | 33.50 | 37.50 |
| 3 | 53.75 | 39.25 | 31.75 | 57.00 | 41.50 | 33.00 |
| C1 | 47.00 | 37.50 | 30.25 | 48.50 | 42.50 | 36.00 |
| 4 | 50.25 | 44.25 | 26.75 | 57.00 | 42.75 | 27.00 |
| C1 | 44.25 | 38.50 | 27.75 | 45.00 | 37.25 | 28.50 |

### Sub-Bandage Pressure Testing (2)

One set of 12 in-house, healthy volunteers were used for comparative testing of sub-bandage pressure of compression bandaging systems of Examples 2, 5, 6 and comparative C1 in sequence. As in the first series of sub-bandage pressure testing, the compression bandage system was applied to the non-dominant leg of the volunteer. As in the previous testing, the first system was applied, tested, removed leaving the pressure transducers untouched in their in their secured positions, transducers re-zeroed, next system applied, tested, removed, etc. until the fourth system was applied and tested. The mean for the sub-bandage pressure measurement results for all 12 volunteers were computed and are reported in Table 5.

TABLE 5

| Example | Sensor 1 | Sensor 2 | Sensor 3 | Sensor 1 | Sensor 2 | Sensor 3 |
|---|---|---|---|---|---|---|
| No. | Rest (mm Hg) | Rest (mm Hg) | Rest (mm Hg) | Walk (mm Hg) | Walk (mm Hg) | Walk (mm Hg) |
| 2 | 43.75 | 49.50 | 24.91 | 48.17 | 56.50 | 29.58 |
| 5 | 46.91 | 51.67 | 31.83 | 48.0 | 57.33 | 37.50 |
| 6 | 51.33 | 61.33 | 36.83 | 55.92 | 69.58 | 42.58 |
| C1 | 51.08 | 49.17 | 31.25 | 54.17 | 55.67 | 39.50 |

### Slippage Measurement of Compression Bandage System

Four sets of 12 in-house, healthy volunteers were used for four sets of comparative testing of downward slippage after 24 h of wearing applied bandage. Compression bandaging systems of Example 5, 6, 7 and C1 were compared in each case to compression bandaging system of Example 2. In order to provide a direct comparison, the bandage system of Example 2 was applied to one leg of the volunteer, and the second bandage system, Examples 5, 6, 7 or C1 respectively, was applied to the other leg. The mean for the slippage results for all 12 volunteers were computed and are reported in Table 6.

TABLE 6

| Example No. | Slippage after 24 h wear (cm) |
|---|---|
| 2 | 0.32 |
| 5 | 0.41 |
| 2 | 0.34 |
| 6 | 0.50 |
| 2 | 0.48 |
| 7 | 0.81 |
| 2 | 0.38 |
| C1 | 4.18 |

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A bandage comprising:
a primary layer comprising a continuous foam substrate;
a secondary layer comprising a fibrous substrate configured to overlay the primary layer; and
a plurality of pressure-differentiating features,
wherein the plurality of pressure-differentiating features are effective to provide localized areas of increased pressure when under compression.

2. (canceled)

3. The bandage of claim 1, characterized by one or more of:
having a relaxed length of about 1 m to about 5 m, and
having a width 40 mm to about 180 mm.

4. (canceled)

5. The bandage of claim 1, wherein the foam substrate is one or more of:
comprised of open-cell foam, closed-cell foam, or a combination thereof, and
selected from polyurethane, carboxylated butadiene-styrene rubber, polyester, polyacrylate, polyether, polyolefin, polychloroprene, silicone, and a combination thereof.

6. (canceled)

7. The bandage of any one of claim 1, wherein one or more of:
the primary layer has a width of at least about 30% of the width of the secondary layer, and
the primary layer has an average thickness of at least 1.6 mm.

8. (canceled)

9. The bandage of claim 1, the primary layer further comprising one or more of:
at least a portion of the plurality of pressure-differentiating features on one or more surfaces, and
at least a portion of the plurality of pressure-differentiating features within the foam substrate.

10. The bandage of claim 1, wherein at least a portion of the plurality of pressure-differentiating features are defined by areas within the foam substrate having a greater thickness.

11. (canceled)

12. The bandage of claim 1, wherein the fibrous substrate is one or more of:
comprised of woven material, knitted material, warp-knit material, nonwoven material, or a combination thereof, and
selected from polyester, rayon, polyethyl acrylate, polyether-polyurea copolymer, and a combination thereof.

13. (canceled)

14. The bandage of claim 1, wherein the fibrous substrate comprises one or more of:
a polymeric binder, and
elastic filaments.

15. (canceled)

16. The bandage of claim 1, wherein the fibrous substrate comprises two fibrous mats selected from woven and nonwoven, elastic filaments between the fibrous mats, and a polymeric binders selected from natural rubber latex, synthetic latex, acrylics, polyethyl acrylate, butadienes, styrene-butadiene rubbers, chloroprenes, ethylenes, isoprenes, nitrile, urethanes, and a combination thereof.

17. (canceled)

18. The bandage of claim 1, wherein the fibrous substrate is one or more of:
self-adhering, and
elastic.

19.-20. (canceled)

21. The bandage of claim 1, the secondary layer comprising one or more of:
at least a portion of the plurality of pressure-differentiation features on one or more surfaces, and
at least a portion of the plurality of pressure-differentiation features are within the fibrous substrate.

22. The bandage of claim 1, wherein at least a portion of the plurality of pressure-differentiating features are defined by areas within the fibrous substrate having a greater thickness.

23. (canceled)

24. The bandage of claim 1, wherein at least a portion of the plurality of pressure-differentiating features are integrated between the primary layer and the secondary layer.

25. The bandage of claim 1, wherein at least a portion of the plurality of pressure-differentiating features are constructed from fibers, stitched fibers, yarns, fabrics, polymeric hot melts, foams, beads, or a combination thereof.

26.-27. (canceled)

28. The bandage of claim 1, wherein at least a portion of the plurality of pressure-differentiating features are effective to provide an increase in sub-bandage pressure from about 10 mmHg to about 120 mmHg with respect to portions of the bandage that are free of pressure-differentiating features when the bandage.

29. The bandage of claim 1, wherein at least a portion of the plurality of pressure-differentiating features are arranged such that the pressure-differentiating features together form a pattern selected from cross-hatch, brick, diamond lattice, hexagonal lattice, stacked equilateral triangles, packed circles, rows, chevron, zigzag, wavy chevron, waves, and a combination thereof.

30. The bandage of claim 1, wherein the plurality of pressure-differentiating features one or more of:
extends over at least 30% of the width of the bandage, and
extends over no more than 50% of the width of the bandage.

31.-32. (canceled)

33. A method for reducing or preventing swelling in an extremity, the method comprising:
providing a bandage of claim 1;
wrapping at least a portion of the extremity with the bandage, wherein the primary layer contacts a skin surface.

34.-44. (canceled)

45. A kit comprising:
a bandage of claim 1; and
a compression substrate; and
a set of instructions directing a user to wrap an extremity with the bandage and the compression substrate.

46. (canceled)

47. A method of preparing at least a portion of a bandage of claim 1, the method comprising:
providing a continuous foam substrate;
folding portions of the continuous foam substrate to provide folded segments;
adhering the folded segments to form at least a portion of the plurality of pressure-differentiating features.

48.-51. (canceled)
